SEQUENCE LISTING (10 sequences)

SEQ ID NO: 1 - DNA, 20 bp, Artificial Sequence
  PSAT1-<L>
gtccaagccagtggatgttt

SEQ ID NO: 2 - DNA, 20 bp, Artificial Sequence
  PSAT1-<R>
tatacagagaggcccggatg

SEQ ID NO: 3 - DNA, 20 bp, Artificial Sequence
  ATAD2-<L>
tatggatggattggacagca

SEQ ID NO: 4 - DNA, 20 bp, Artificial Sequence
  ATAD2-<R>
agatctgtgggtagcgtcgt

SEQ ID NO: 5 - DNA, 20 bp, Artificial Sequence
  ASB9-<L>
catcatggatggcaaacaag

SEQ ID NO: 6 - DNA, 20 bp, Artificial Sequence
  ASB9-<R>
gctgtcacaccattcacctg

SEQ ID NO: 7 - DNA, 20 bp, Artificial Sequence
  SLC7A11-<L>
ggcagtgaccttttctgagc

SEQ ID NO: 8 - DNA, 20 bp, Artificial Sequence
  SLC7A11-<R>
aactgccagcccaataaaaa

SEQ ID NO: 9 - DNA, 20 bp, Artificial Sequence
  CKAP2L-<L>
aaacggcctcctatggaact

SEQ ID NO: 10 - DNA, 20 bp, Artificial Sequence
  CKAP2L-<R>
tattggtgttgccccatttt

(19) United States
(12) Patent Application Publication
(10) Pub. No.: US 2011/0201512 A1
(43) Pub. Date: Aug. 18, 2011

(54) Biomarkers Indicative of Colon Cancer and Metastasis and Diagnosis and Screening Therapeutics Using the Same

(75) Inventors: Hee Gu Lee, Daejeon (KR); Eun Young Song, Daejeon (KR); Young II Yeom, Daejeon (KR); Jae Wha Kim, Daejeon (KR); Jong Tae Kim, Daejeon-si (KR); Kyung Sook Chung, Daejeon (KR); Mi Sun Won, Daejeon (KR); Hyun Hyo Suh, Jinju-si (KR); Joo Woong Park, Seoul (KR); Min Ah Kang, Gumi-si (KR)

(73) Assignee: Korea Research Institute of Bioscience and Biotechnology, Daejeon (KR)

(21) Appl. No.: 12/822,556

(22) Filed: Jun. 24, 2010

(30) Foreign Application Priority Data
Jun. 25, 2009 (KR) 10-2009-0056874

(51) Int. Cl. C40B 30/04; C12Q 1/68; G01N 33/574

(52) U.S. Cl. 506/9; 435/6.12; 436/501; 435/7.1

(57) ABSTRACT

The present invention relates to a method for detecting a colon cancer in a human, comprising the steps of: (a) providing a biological sample from the human; and (b) detecting the level of a ATAD2 nucleic acid or a ATAD2 protein in the biological sample, relative to the level of the ATAD2 nucleic acid or the ATAD2 protein in a control sample from a normal human, wherein an increased level of the ATAD2 nucleic acid or the ATAD2 protein in the biological sample compared to the control sample indicates that the human has the colon cancer. The biomarker of this invention was identified using normal colon tissue, colon cancer tissue and metastatic cancer tissue derived from a colon cancer patient. Therefore, the accuracy and reliability of the present biomarker for colon cancer and/or metastasis are much more significantly improved. In addition, the biomarker of this invention permits to identify and predict colon cancer or metastasis in an accurate manner.

### CROSS-REFERENCE TO RELATED APPLICATIONS

This application claims priority from Korean Patent Application No. 2009-0056874, filed on Jun. 25, 2009, in the Korean Intellectual Property Office, the disclosure of which is incorporated herein by reference.

### BACKGROUND OF THE INVENTION

1. Field of the Invention

The present invention relates to biomarkers specific to colon cancer and/or metastasis, and uses thereof.

2. Description of the Related Art

Colon has some characteristics as follows: (a) container of food waste after digestion and absorption of food; (b) formation of the feces through absorption of all remaining water from the food waste; and (c) harbor of several kinds of bacteria. Colon is approximately 2 m in length and is composed of colorectum, rectum and anus. Colon cancer may be developed in any tissue containing colonic mucosa. First of all, sigmoid colon and rectum are known to be the tissue in which colon cancer is frequently developed.

Presently, the incidence rate of colon cancer has been much more remarkably enhanced in Korea. The death caused by colon cancer in men holds 4th position next to stomach cancer, lung cancer and liver cancer, and also similarly in women. It was investigated that the incidence frequency of colon cancer in men is higher than that in women. Colon cancer is developed most frequently in 50 y of age, followed by 60 y of age. Compared the incidence frequency of colon cancer in Korea with that in EP or USA, it is likely that the age-specific incidence rate of colon cancer in Korea is about 10 years younger than that in EP or USA. The incidence rate of colon cancer is generated in 30 y of age with a frequency of 5-10%. In addition, the development of colon cancer in young people shows a hereditary tendency to be frequently developed in family.

It has been supposed that environmental factor in the development of colon cancer is more important than hereditary factor. For example, excessive intake of animal lipid or protein from rapid westernization of dietary life is considered as a causative factor. However, it has been known that approximately 5% of colon cancer is developed by hereditary factors.

The risk group in which colon cancer is feasibly developed is as follows: (a) the group having an experience on colon polyps; (b) the group having family history; (c) the group suffering from long-term ulcerative colitis; (d) the group having almost incurable anal fistula.

As a representative to classify colon cancers, there are Dukes classification and UICC stage classification method. The standard of colon classification is provided according to the extent of depth that cancer is invaded into bowel wall and occurrence of distant metastasis, not cancer size (5-year survival rate after surgery depending on each stage is described in parenthesis).

[Dukes Classification Method]
Dukes A (not less than 90%): cancer is invaded into but not through the bowel wall.
Dukes B (60-80%): cancer is penetrated through the bowel wall, but lymph node metastasis is not found.
Dukes C (20-50%): involvement of lymph node metastasis.
Dukes D (not more than 20%): distant metastasis is found in peritoneum, liver, lung, etc.

[Stage Classification Method]
Stage 0: cancer is limited to mucosa.
Stage 1: cancer is limited to the bowel wall.
Stage 2: cancer is penetrated through the bowel wall, but not extended to adjacent organs.
Stage 3: cancer is penetrated through adjacent organs, or lymph node metastasis is found.
Stage 4: distant metastasis is found in peritoneum, liver, lung, etc.

In general, it has been accepted that each Dukes A, Dukes B, Dukes C and Dukes D corresponds to stage 0 and 1, stage 2, stage 3, and stage 4, in spite of a few differences between the afore-mentioned classification methods. Particularly, Dukes classification method is utilized in a world-wide manner.

Where colon cancer is detected in an early stage, it may be completely treated by endoscopic application or surgical treatment. It may be expected that colon cancer is perfectly treated with surgery operation where it is identified in operable stage irrespective of metastatic stage to liver or lung. In other words, surgical treatment is so far the most effective therapeutic method. Accordingly, early diagnosis and treatment of colon cancer is indispensable because colon cancer may be metastasized to a tissue that surgical resection is difficult where it is identified in a late stage.

Colon cancer recurrence is very common after surgery, and thus a medical checkup at regular (interval of 3-4 months) has to be carried out to examine the presence or absence of recurrence. Liver, lung or peridoneum is an organ which is likely to be recurrent. In addition, colon cancer is likely to be locally recurrent in a surgical region. Colon cancer may be perfectly cured through resection of recurrence lesion where recurrence of colon cancer is identified in an early period compared to that of other cancers. As recurrence of not less than 80% is found within three years at post-surgery, it is the standard of complete recovery to find no recurrence within five years at post-surgery.

Colon cancer may be healed up to almost 100% in an early stage. However, a regular checkup has to be carried out because it is very difficult to detect colon cancer in no symptom period, which is generally developed under the absence of subjective symptom.

The hemoccult test is a representative of colon cancer screening. However, the positive response of the hemoccult test does not necessarily indicate the development of colon cancer, vice versa.

The colon cancer tests used presently are as follows:

(a) Colonography

The morphology of intestine is imaged using X-ray photograph through injection of barium and air to anus after the bowel is sufficiently cleared by dietary restriction.

(b) Colonoscopy

Currently, colonoscopy is divided into short endoscopy and long endoscopy, enabling to observe up to sigmoid colon (S-colon) and all colons, respectively. Colonoscopy procedure is more accurate than colonography procedure because both colon cancer testing and resection of polyps are able to be carried out simultaneously.

(c) Tumor Marker

It is a method to identify hidden cancers of any part in body using blood test. However, there has been no tumor marker capable of being utilized for identification of colon cancer in an early stage. CEA (Carcinoembryonic antigen) is a general marker, but only half of colon cancer is detected as a positive signal. Therefore, it is utilized as an indicator to evaluate colon cancer progression and therapeutic efficacy.

(d) CT (Computerized Tomography), MRI (Magnetic Resonance Imaging), Sonography

All diagnostic methods are much more advanced tests to detect cancers of other parts in body, but not suitable to examine colon-related diseases. They have been utilized in colon cancer diagnosis to determine to what extent primary lesion is progressed and distant metastasis to liver is generated.

Representative examples known as molecular markers of colon cancer are as follows:

WO 2005/015224 discloses a method to diagnose colorectal cancer using an antibody against protein RLA-0 (60S acidic ribosomal protein P0). WO 2004/079368 discloses that HSP90 is highly expressed in colorectal cancers. WO 2004/071267 describes a method for diagnosing colorectal cancer in an early stage by measuring NNMT (nicotinamide N-methyltransferase) in a stool sample, and WO 2005/015234 discloses that SAHH (S-adenosylhomocysteine hydrolase) protein is able to be utilized in the diagnosis of colorectal cancer. In addition, U.S. Pat. No. 7,501,243 discloses TTK (Tyrosine threonine kinase) as a colon cancer marker.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive studies to develop a novel biomarker for identifying colon cancer and/or metastasis at a molecular level in a high-throughput and accurate manner. As results, we have discovered biomarkers capable of early detecting and predicting colon cancer and/or metastasis.

Accordingly, it is an object of this invention to provide a method for detecting a colon cancer in a human.

It is still another object of this invention to provide a screening method of a substance for preventing or treating colon cancer.

Other objects and advantages of the present invention will become apparent from the following detailed description together with the appended claims and drawings.

### BRIEF DESCRIPTION OF THE DRAWINGS

FIGS. 1a-1b are to analyze the expression pattern of 2,230 genes in normal colon cancer tissue, colon cancer tissue and metastatic tissue using a 48K human microarray chip (Illumina Inc.) (p value<0.05). “DF” and “RC” before the number indicate normal tissue and recurrent colon cancer tissue, respectively. The gene expression is sharply enhanced in order of red, chestnut, black, yellowish green, dark green, green, bright green and yellow-green color.

FIG. 2 represents the expression pattern of genes expressed highly in colon cancer tissues.

FIG. 3 represents RT-PCR analyzing the expression of colon cancer biomarkers of the present invention in normal and colon cancer tissue. Odd lanes indicate RT-PCR pattern in normal tissue; and even lanes indicate RT-PCR pattern in colon cancer tissue.

FIG. 4 is a gel image analyzing the expression of colon cancer biomarkers of the present invention in colon cancer cell lines using RT-PCR. Lane 1: DLD-1; Lane 2: HT29; Lane 3: HCT116; Lane 4: colo205; Lane 5: SW480; Lane 6: SW620; Lane 7: SNU-C1: Lane 8: SNU-C2A; Lane 9: KM12C; Lane 10: KM12SM.

FIG. 5 show a Western blotting analyzing the expression pattern of ATAD2 protein in DLD-1, HT29, HCT116, colo205, SW480, SW620, SNU-C1 and KM12C cell lines.

FIG. 6 represents the expression of the colon cancer biomarker (ATAD2) of the present invention using an immunohistochemical staining in normal and colon cancer tissues.

### DETAILED DESCRIPTION OF THIS INVENTION

In one aspect of this invention, there is provided a method for detecting a colon cancer in a human, comprising the steps of: (a) providing a biological sample from the human; and (b) detecting the level of a ATAD2 (ATPase family, AAA domain containing 2) nucleic acid or a ATAD2 protein in the biological sample, relative to the level of the ATAD2 nucleic acid or the ATAD2 protein in a control sample from a normal human, wherein an increased level of the ATAD2 nucleic acid or the ATAD2 protein in the biological sample compared to the control sample indicates that the human has the colon cancer.

The present inventors have done intensive studies to develop a novel biomarker for identifying colon cancer at a molecular level in a high-throughput and accurate manner. As results, we have discovered biomarkers capable of early detecting and predicting colon cancer and/or metastasis. In particular, biomarkers of this invention were identified using normal colorectal tissue, colorectal cancer tissue and metastatic cancer tissue derived from a colon cancer patient. Therefore, accuracy and reliability of the present biomarkers for colon cancer were much more significantly improved.

The term “colon cancer” means a name including rectal cancer, colon cancer and anal cancer.

The molecular marker of this invention may be indicative of colon cancer development, progression and/or metastasis, and also used in diagnosis of colon cancer development, progression and/or metastasis.

The term “detecting a cancer” used herein includes the following matters: (a) to determine susceptibility of a subject to a particular disease or disorder; (b) to evaluate whether a subject has a particular disease or disorder; (c) to assess a prognosis of a subject suffering from a specific disease or disorder (e.g., identification of pre-metastatic or metastatic cancer conditions, determination of cancer stage, or investigation of cancer response to treatment); or (d) therametrics (e.g., monitoring conditions of a subject to provide an information to treatment efficacy).

In the present invention, a biological sample is provided from humans. The biological sample useful in the present invention includes any sample. The biological sample can be derived from a particular tissue or organ, particularly colon epithelium. The biosample to be analyzed may include any cell, tissue, or fluid (blood, serum and plasma) from a biological source.

Following preparation of biological samples, a ATAD2 nucleic acid or a ATAD2 protein in the biological sample is detected, relative to the level of the ATAD2 nucleic acid or the ATAD2 protein in a control sample from a normal human, wherein an increased level of the ATAD2 nucleic acid or the ATAD2 protein in the biological sample compared to the control sample indicates that the human has the colon cancer.

A “control sample” refers to a sample of biological material representative of healthy, cancer-free animals, and/or cells or tissues. The level of ATAD2 in a control sample is desirably typical of the general population of normal, cancer-free animals or of a particular individual, or in a particular tissue. A control sample can also refer to an established level of ATAD2, representative of the cancer-free population, that has been previously established based on measurements from normal, cancer-free animals.

An “increased level of ATAD2” means a level of ATAD2, that, in comparison with a control level of ATAD2, is detectably higher. The method of comparison can be statistical, using quantified values for the level of ATAD2, or can be compared using non-statistical means, such as by visual assessment by a human.

According to a preferable embodiment, the detection of the step (b) in the invention may be carried out by analyzing the level of an mRNA of ATAD2.

According to a preferable embodiment, the analysis of the level of an mRNA of ATAD2 may be carried out by a microarray.

Using microarray in the method of the present invention, probes are immobilized on the solid surface of microarray. The method of the present invention utilizing gene amplification includes primers.

Probes or primers used in the method of the present invention have a sequence complementary to a nucleotide sequence selected from the group consisting of PSAT1, ATAD2, ASB9, SLC7A11 and CKAP2L, and most preferably ATAD2. The term “complementary” with reference to sequence used herein refers to a sequence having complementarity to the extent that the sequence hybridizes or anneals specifically with the nucleotide sequence described above under certain hybridization or annealing conditions. In this regard, the term “complementary” used herein has different meaning from the term “perfectly complementary”. The primer or probe of this invention may include one or more mismatch base sequences where it is able to specifically hybridize with the above-described nucleotide sequences.

The term “primer” used herein means a single-stranded oligonucleotide which is capable of acting as a point of initiation of template-directed DNA synthesis when placed under proper conditions (i.e., in the presence of four different nucleoside triphosphates and a thermostable enzyme) in an appropriate buffer and at a suitable temperature. The suitable length of primers will depend on many factors, including temperature, application and source of primer, generally, 15-30 nucleotides in length. In general, shorter primers need lower temperature to form stable hybridization duplexes to templates.

The sequences of primers are not required to have perfectly complementary sequence to templates. The sequences of primers may comprise some mismatches, so long as they can be hybridized with templates and serve as primers. Therefore, the primers of this invention are not required to have perfectly complementary sequence to the nucleotide sequence as described above; it is sufficient that they have complementarity to the extent that they anneals specifically to the nucleotide sequence of the gene for acting as a point of initiation of synthesis. The primer design may be conveniently performed with referring to the above-described nucleotide sequences. For instance, the primer design may be carried out using computer programs for primer design (e.g., PRIMER 3 program).

The term “probe” used herein refers to a linear oligomer of natural or modified monomers or linkages, including deoxyribonucleotides, ribonucleotides and the like, which is capable of specifically hybridizing with a target nucleotide sequence, whether occurring naturally or produced synthetically. The probe used in the present method may be prepared in the form of preferably single-stranded and oligodeoxyribonucleotide probe.

To prepare primers or probes, the nucleotide sequence of the present biomarker may be found in the GenBank. For example, the nucleotide sequences of PSAT1 (phosphoserine aminotransferase 1), ATAD2 (ATPase family, MA domain containing 2), ASB9 (ankyrin repeat and SOCS box-containing 9), SLC7A11 (solute carrier family 7, (cationic amino acid transporter, y+ system) member 11) and CKAP2L (cytoskeleton associated protein 2-like) as the biomarker of this invention are disclosed in GenBank Accession Nos. NM_{—}021154.3, NM_{—}014109.3, NM_{—}001031739.1, NM_{—}014331.3 and NM_{—}152515.3, respectively, and primers or probes may be designed by reference with the nucleotide sequence afore-mentioned.

In microarray, the present probes serve as a hybridizable array element and are immobilized on a substrate. A preferable substrate includes suitable solid or semi-solid supporters, such as membrane, filter, chip, slide, wafer, fiber, magnetic or nonmagnetic bead, gel, tubing, plate, macromolecule, microparticle and capillary tube. The hybridizable array elements are arranged and immobilized on the substrate. Such immobilization occurs through chemical binding or covalent binding such as UV. In an embodiment of this invention, the hybridizable array elements are bound to a glass surface modified to contain epoxy compound or aldehyde group or to a polylysin-coated surface using UV. Further, the hybridizable array elements are bound to a substrate through linkers (e.g., ethylene glycol oligomer and diamine).

DNAs to be examined with a microarry of this invention may be labeled, and hybridized with array elements on microarray. Various hybridization conditions are applicable, and for the detection and analysis of the extent of hybridization, various methods are available depending on labels used.

The present method for identifying colon cancer and/or metastasis may be carried out in accordance with hybridization. For such analysis, probes, which have a complementary sequence to the nucleotide sequence of the biomarkers of this invention as set forth, are used.

Using probes hybridizable with the nucleotide sequence of the biomarkers of this invention, colon cancer may be determined by hybridization-based assay.

Labels linking to the probes may generate a signal to detect hybridization and bound to oligonucleotide. Suitable labels include fluorophores (e.g., fluorescein, phycoerythrin, rhodamine, lissamine, Cy3 and Cy5 (Pharmacia)), chromophores, chemiluminescents, magnetic particles, radioisotopes (e.g., P³² and S³⁵), mass labels, electron dense particles, enzymes (e.g., alkaline phosphatase or horseradish peroxidase), cofactors, substrates for enzymes, heavy metals (e.g., gold), and haptens having specific binding partners, e.g., an antibody, streptavidin, biotin, digoxigenin and chelating group, but not limited to. Labeling is performed according to various methods known in the art, such as nick translation, random priming (Multiprime DNA labeling systems booklet, “Amersham” (1989)) and kination (Maxam & Gilbert, Methods in Enzymology, 65: 499 (1986)). The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal.

The nucleic acid sample to be analyzed may be prepared using mRNA from various biosamples. Preferably, the biosample is colon cells. Instead of probes, cDNA of interest may be labeled for hyribridization-based analysis.

Probes are hybridized with cDNA molecules under stringent conditions. Suitable hybridization conditions may be routinely determined by optimization procedures. To establish a protocol for use of laboratory, these procedures may be carried out by various methods known to those ordinarily skilled in the art. Conditions such as temperature, concentration of components, hybridization and washing times, buffer components, and their pH and ionic strength may be varied depending on various factors, including the length and GC content of probes and target nucleotide sequence. The detailed conditions for hybridization can be found in Joseph Sambrook, et al., Molecular Coning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). For example, the high stringent condition includes hybridization in 0.5 M NaHPO₄, 7% SDS (sodium dodecyl sulfate) and 1 mM EDTA at 65° C. and washing in 0.1×SSC (standard saline citrate)/0.1% SDS at 68° C. Also, the high stringent condition includes washing in 6×SSC/0.05% sodium pyrophosphate at 48° C. The low stringent condition includes e.g., washing in 0.2×SSC/0.1% SDS at 42° C.

Following hybridization reactions, a hybridization signal indicative of the occurrence of hybridization is then measured. The hybridization signal may be analyzed by a variety of methods depending on labels. For example, where probes are labeled with enzymes, the occurrence of hybridization may be detected by reacting substrates for enzymes with hybridization resultants. The enzyme/substrate pair useful in this invention includes, but is not limited to, a pair of peroxidase (e.g., horseradish peroxidase) and chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) and naphtol/pyronine; a pair of alkaline phosphatase and bromochloroindolylphosphate (BCIP), nitro blue tetrazolium (NBT), naphthol-AS-B1-phosphate and ECF substrate; and a pair of glucose oxidase and t-NBT (nitroblue tetrazolium) or m-PMS (phenzaine methosulfate). Where probes are labeled with gold particles, the occurrence of hybridization may be detected by silver staining method using silver nitrate. In these connections, where the present method for identifying colon cancer is carried out by hybridization, it comprises the steps of: (i) hybridizing a nucleic acid sample to a probe having a nucleotide sequence complementary to the nucleotide sequence of the biomarker of this invention as set forth; and (ii) detecting the occurrence of hybridization. The signal intensity from hybridization is indicative of colon cancer. When the hybridization signal to the biomarker of this invention from a sample to be diagnosed is measured to be stronger than normal samples (e.g., normal colon epithelial cells), the sample can be determined to have colon cancer.

According to a preferable embodiment, the method of this invention may be carried out by various gene amplification procedures. More preferably, the analysis of the level of an mRNA of ATAD2 may be carried out by RT-PCR (reverse transcription-polymerase chain reaction).

The term used herein “amplification” refers to reactions for amplifying nucleic acid molecules. A multitude of amplification reactions have been suggested in the art, including polymerase chain reaction (hereinafter referred to as PCR) (U.S. Pat. Nos. 4,683,195, 4,683,202, and 4,800,159), reverse transcription-polymerase chain reaction (hereinafter referred to as RT-PCR) (Sambrook, J. et al., Molecular Cloning. A Laboratory Manual, 3rd ed. Cold Spring Harbor Press (2001)), the methods of Miller, H. I. (WO 89/06700) and Davey, C. et al. (EP 329,822), ligase chain reaction (LCR), Gap-LCR (WO 90/01069), repair chain reaction (EP 439,182), transcription-mediated amplification (TMA; WO 88/10315), self sustained sequence replication (WO 90/06995), selective amplification of target polynucleotide sequences (U.S. Pat. No. 6,410,276), consensus sequence primed polymerase chain reaction (CP-PCR; U.S. Pat. No. 4,437,975), arbitrarily primed polymerase chain reaction (AP-PCR; U.S. Pat. Nos. 5,413,909 and 5,861,245), nucleic acid sequence based amplification (NASBA; U.S. Pat. Nos. 5,130,238, 5,409,818, 5,554,517 and 6,063,603), strand displacement amplification and loop-mediated isothermal amplification (LAMP), but not limited to. Other amplification methods that may be used are described in U.S. Pat. Nos. 5,242,794, 5,494,810, 4,988,617 and in U.S. Ser. No. 09/854,317.

PCR is one of the most predominant processes for nucleic acid amplification and a number of its variations and applications have been developed. For example, for improving PCR specificity or sensitivity, touchdown PCR, hot start PCR, nested PCR and booster PCR have been developed with modifying traditional PCR procedures. In addition, real-time PCR, differential display PCR (DD-PCR), rapid amplification of cDNA ends (RACE), multiplex PCR, inverse polymerase chain reaction (IPCR), vectorette PCR and thermal asymmetric interlaced PCR (TAIL-PCR) have been suggested for certain applications. The details of PCR can be found in McPherson, M. J., and Moller, S. G. PCR. BIOS Scientific Publishers, Springer-Verlag New York Berlin Heidelberg, N.Y. (2000), the teachings of which are incorporated herein by reference in its entity.

Where the present method for identifying colon cancer is carried out using primers, the nucleic acid amplification is executed for analyzing the expression level of the nucleotide sequence of the present biomarkers. Because the present invention is intended to assess the expression level of the nucleotide sequence of the present biomarkers, their mRNA levels in samples (e.g., colon epithelial cells) ares analyzed.

Therefore, the present invention may be generally carried out by nucleic acid amplifications using mRNA molecules in samples as templates and primers to be annealed to mRNA or cDNA.

For obtaining mRNA molecules, total RNA is isolated from samples. The isolation of total RNA may be performed by various methods (Sambrook, J. et al., Molecular Cloning. A Laboratory Manual, 3rd ed. Cold Spring Harbor Press (2001); Tesniere, C. et al., Plant Mol. Biol. Rep., 9: 242 (1991); Ausubel, F. M. et al., Current Protocols in Molecular Biology, John Willey & Sons (1987); and Chomczynski, P. et al., Anal. Biochem. 162: 156 (1987)). For example, total RNA in cells may be isolated using Trizol. Afterwards, cDNA molecules are synthesized using mRNA molecules isolated and then amplified. Since total RNA molecules used in the present invention are isolated from human samples, mRNA molecules have poly-A tails and converted to cDNA by use of dT primer and reverse transcriptase (PNAS USA, 85: 8998 (1988); Libert F, et al., Science, 244: 569 (1989); and Sambrook, J. et al., Molecular Cloning. A Laboratory Manual, 3rd ed. Cold Spring Harbor Press (2001)). cDNA molecules synthesized are then amplified by amplification reactions.

The primers used for the present invention is hybridized or annealed to a region on template so that double-stranded structure is formed. Conditions of nucleic acid hybridization suitable for forming such double stranded structures are described by Joseph Sambrook, et al. Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E coli DNA polymerase I, a thermostable DNA polymerase and bacteriophage T7 DNA polymerase. Preferably, the polymerase is a thermostable DNA polymerase obtained from a variety of bacterial species, including Thermus aquaticus (Taq), Thermus thermophilus (Tth), Thermus filiformis, Thermis flavus, Thermococcus literatis, and Pyrococcus furiosus (Pfu).

When a polymerization reaction is being conducted, it is preferable to provide the components required for such reaction in excess in the reaction vessel. Excess in reference to components of the amplification reaction refers to an amount of each component such that the ability to achieve the desired amplification is not substantially limited by the concentration of that component. It is desirable to provide to the reaction mixture an amount of required cofactors such as Mg²⁺, and dATP, dCTP, dGTP and dTTP in sufficient quantity to support the degree of amplification desired. All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present invention is performed under stringent conditions that allow for specific binding between the target nucleotide sequence and the primer. Such stringent conditions for annealing will be sequence-dependent and varied depending on environmental parameters.

The amplified cDNA to the nucleotide sequence of the biomarkers of this invention are then analyzed to assess their expression level using suitable methods. For example, the amplified products are resolved by a gel electrophoresis and the bands generated are analyzed to assess the expression level of the nucleotide sequence of the present biomarkers. When the expression level of the nucleotide sequence of the present biomarkers from a sample to be diagnosed is measured to be higher than normal samples (normal cells), the sample can be determined to have colon cancer/metastasis.

In these connections, where the present method for identifying colon cancer biomarkers is carried out by amplification reactions, it comprises the steps of: (i) amplifying a nucleic acid sample by use of a primer to be annealed to the nucleotide sequence of the present biomarkers as set forth; and (ii) analyzing the amplified products to determine the expression level of the nucleotide sequence of the present biomarkers.

According to a preferable embodiment, the detection of the step (b) in the invention may be carried out by analyzing the level of the ATAD2 protein. More preferably, the analysis of the level of the ATAD2 protein may be carried out by an immunoassay, i.e. antigen-antibody reactions. In this content, this invention may be performed using an antibody or aptamer binding specifically to the present colon biomarkers.

The antibody against the biomarkers used in this invention may polyclonal or monoclonal, preferably monoclonal. The antibody could be prepared according to conventional techniques such as a fusion method (Kohler and Milstein, European Journal of Immunology, 6: 511-519 (1976)), a recombinant DNA method (U.S. Pat. No. 4,816,56) or a phage antibody library (Clackson, et al, Nature, 352: 624-628 (1991) and Marks, et al, J. Mol. Biol., 222: 58, 1-597 (1991)). The general procedures for antibody production are described in Harlow, E. and Lane, D., Antibodies: A Laboratory Manual, Cold Spring Harbor Press, New York, 1988; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, which are incorporated herein by references. For example, the preparation of hybridoma cell lines for monoclonal antibody production is done by fusion of an immortal cell line and the antibody-producing lymphocytes. This can be done by techniques well known in the art. Polyclonal antibodies may be prepared by injection of the protein antigen to suitable animal, collecting antiserum containing antibodies from the animal, and isolating specific antibodies by any of the known affinity techniques.

Where the method of this invention is performed using antibodies or aptamers to the biomarker proteins, it could be carried out according to conventional immunoassay procedures for identifying colon cancer/metastasis.

Such immunoassay may be executed by quantitative or qualitative immunoassay protocols, including radioimmunoassay, radioimmuno-precipitation, immunoprecipitation, immunostaining assay, enzyme-linked immunosorbent assay (ELISA), capture-ELISA, inhibition or competition assay, sandwich assay, flow cytometry, immunofluorescence assay and immuoaffinity assay, but not limited to. The immunoassay and immuostaining procedures can be found in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies: A Laboratory Manual, Cold Spring Harbor Press, 1999, which are incorporated herein by references.

For example, according to the radioimmunoassay method, the radioisotope (e.g., C¹⁴, I¹²⁵, P³² and S³⁵)-labeled antibody may be used to detect the biomarker protein of this invention.

In addition, according to the ELISA method, the particular Example of the present invention may comprise the steps of: (i) coating a surface of solid substrates with cell lysates to be analyzed; (ii) incubating the coated cell lysates with a primary antibody against a biomarker protein; (iii) incubating the resultant of step (ii) with a secondary antibody conjugated with an enzyme; and (iv) measuring the activity of the enzyme.

The solid substrate useful in this invention includes carbohydrate polymer (e.g., polystyrene and polypropylene), glass, metal or gel, and most preferably microtiter plates.

The enzyme conjugated with the secondary antibody includes an enzyme which catalyzes colorimetric, fluorometric, luminescence or infra-red reactions, e.g., including alkaline phosphatase, β-galactosidase, horseradish peroxidase, luciferase and cytochrome P₄₅₀, but not limited to. Where using alkaline phosphatase, bromochloroindolylphosphate (BCIP), nitro blue tetrazolium (NBT), naphthol-AS-B1-phosphate and enhanced chemifluorescence (ECF) may be used as a substrate for color-developing reactions; in the case of using horseradish peroxidase, chloronaphtol, aminoethylcarbazol, diaminobenzidine, D-luciferin, lucigenin methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) and naphtol/pyronin, glucose oxidase and tNBT (nitroblue tetrazolium) and m-PMS (phenzaine methosulfate) may be used as a substrate.

According to the capture-ELISA method, the specific Example of the present method may comprise the steps of: (i) coating a surface of a solid substrate with an antibody of a biomarker protein as a capturing antibody; (ii) incubating the capturing antibody with a cell sample; (iii) incubating the resultant of step (ii) with a detecting antibody having a fluorescent label which reacts with the biomarker protein specifically; and (iv) measuring the signal generated from the label.

The detecting antibody includes a substance generating a detectable signal. The signal-generating substance bound to antibody includes, but is not limited to, chemical (e.g., biotin), enzyme (alkaline phosphatase, β-galactosidase, horseradish peroxidase and cytochrome P₄₅₀), radio-isotope (e.g., C¹⁴, I¹²⁵, P³² and S³⁵), fluorescent (e.g., fluorescein), luminescent, chemiluminescent and FRET (fluorescence resonance energy transfer) substances. Various methods for labels and labelings are described in Ed Harlow and David Lane, Using Antibodies: A Laboratory Manual, Cold Spring Harbor Laboratory Press, 1999.

The analysis for measuring the activity or the signal of final enzyme in the ELISA and capture-ELISA method may be carried out by various methods known to those skilled in the art. The signal detection permits to a qualitative or quantitative analysis of the present markers. For example, the signal of each biotin- and luciferase-labeled protein may be feasibly detected using streptavidin and luciferin.

According to another modification of this invention, aptamer having a specific binding affinity to the biomarker of the present invention may be used instead of antibody. Aptamer means an oligonucleic acid or peptide molecule, and general descriptions of aptamer are disclosed in Bock L C et al., Nature 355 (6360): 564-566 (1992); Hoppe-Seyler F, Butz K “Peptide aptamers: powerful new tools for molecular medicine”. J Mol Med. 78 (8): 426-430 (2000); and Cohen B A, Colas P, Brent R. “An artificial cell-cycle inhibitor isolated from a combinatorial library”. Proc Natl Acad Sci USA. 95 (24): 14272-14277 (1998).

The final signal intensity measured by the above-mentioned immunoassay procedures is indicative of colon cancer/metastasis. When the signal to the biomarker of this invention in a sample of interest is stronger than that in normal samples, the sample includes colon cancer/metastasis.

The method of the present invention may optionally include other reagents along with primers, probes or antibodies described above. For instance, where the present method may be used for nucleic acid amplification, it may optionally include the reagents required for performing PCR reactions such as buffers, DNA polymerase (thermostable DNA polymerase obtained from Thermus aquaticus (Taq), Thermus thermophilus (Tth), Thermus filiformis, Thermis flavus, Thermococcus literalis, and Pyrococcus furiosus (Pfu)), DNA polymerase cofactors, and dNTPs. The methods, typically, are adapted to contain in separate packaging or compartments including the constituents afore-described.

The biomarkers of the present invention are biomolecules expressed highly in colon cancer/metastasis. The high expression of biomarkers may be measured at mRNA or protein level. The term “high expression” with reference to colon cancer/metastasis means that the nucleotide sequence of interest in a sample to be analyzed is much more highly expressed than that in the normal sample, for instance, a case analyzed as high expression according to analysis methods known to those skilled in the art, e.g., RT-PCR method or ELISA method (See, Sambrook, J., et al, Molecular Cloning. A Laboratory Manual, 3rd ed. Cold Spring Harbor Press (2001)). Using analysis methods as described above, where the bimarkers of the present invention are much more highly expressed at a range of 2-10 folds in cancer cells than in normal cells, this case is determined as “high expression” and identified as colon cancer/metastasis in the present invention.

Among biomarkers of this invention, PSAT1, ATAD2, ASB9, SLC7A11 and CKAP2L are noticeably utilized in identification of colon cancer metastasis.

In still another aspect of this invention, there is provided a screening method of a substance for preventing or treating colon cancer or metastasis, comprising the steps of:

(a) contacting a sample of interest to a cell containing a nucleotide sequence ATAD2; and

(b) measuring an expression of the nucleotide sequence, wherein the sample is determined as the substance for preventing or treating colon cancer or metastasis with the proviso that the high-expression of the nucleotide sequence is inhibited by the sample.

According to the present method, cells containing the nucleotide sequence of the present biomarker are first contacted to a sample to be analyzed. Preferably, cells containing the nucleotide sequence of the present biomarker are human colon cancer cells. The term “sample” used herein in conjunction with the present screening method refers to a material tested in the present method for analyzing the influence on the expression level of the present biomarker. The sample includes chemical substances, nucleotides, antisense-RNA, siRNA (small interference RNA) and extract of natural source, but not limited to.

Afterwards, the expression level of the present biomarkers in cells is measured. The measurement of their expression levels may be carried out according to methods as described above, and the sample may be determined as the substance for preventing or treating the colon cancer where the high-expression of the nucleotide sequence of the present biomarker is inhibited.

The features and advantages of this invention are summarized as follows:

(a) The present invention provides a novel molecular biomarker for identifying colon cancer and/or metastasis.

(b) The biomarkers of this invention were identified using normal colorectal tissue, colorectal cancer tissue and metastatic cancer tissue derived from a colon cancer patient. Therefore, the accuracy and reliability of the present biomarkers for colon cancer are much more significantly improved.

(c) The biomarker of this invention permits to identify and predict colon cancer in an accurate manner.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### DNA Chip Analysis and Data Mining for Identifying a Colon Cancer and Metastasis-Specific Biomarker

To primarily identify genes which are specifically overexpressed only in colon cancer and/or metastatic cells compared with normal colon epithelial cells, the expression profile of 2,230 genes was examined using a DNA chip (48K human microarray, Illumina Inc.). Initially, normal colon epithelial and colon cancer tissue samples were harvested. Colon cancer tissue samples were obtained from a primary tumor tissue and metastasis tissue (liver metastatic tissue) of a colorectal cancer subject, followed by identifying a molecular biomarker for both colon cancer primary tumor and metastasis.

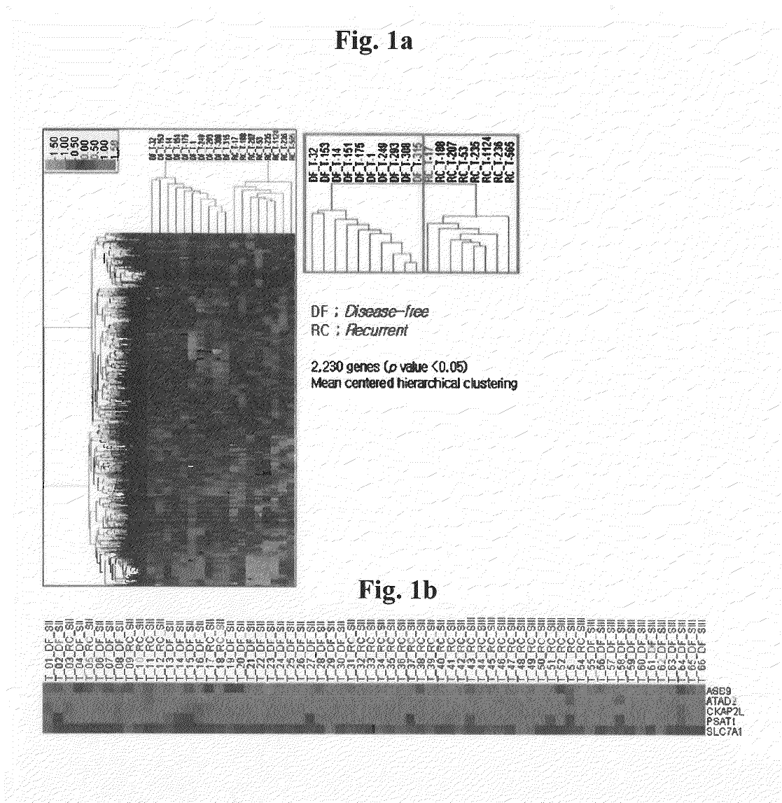

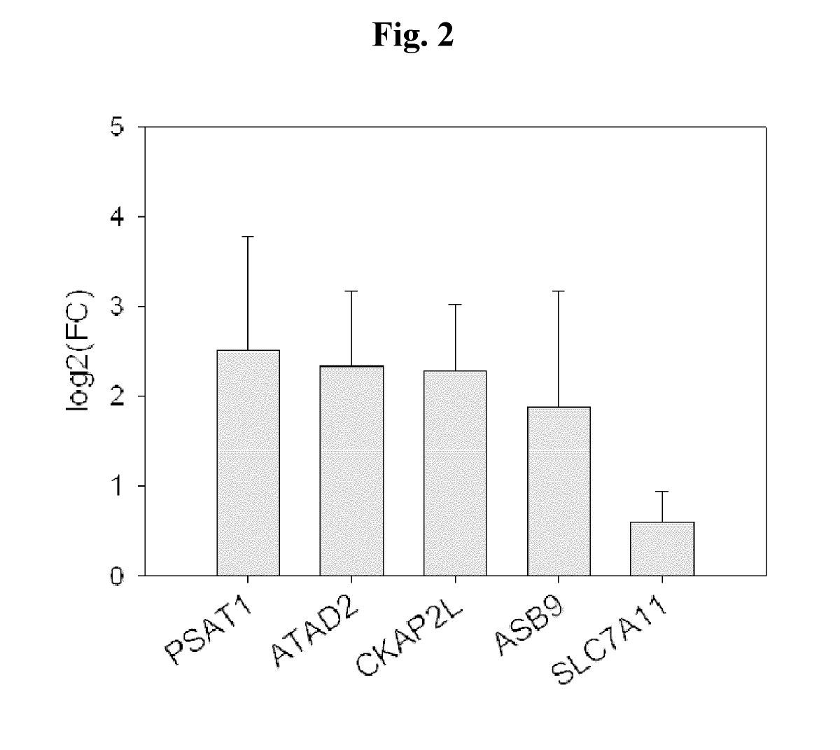

Total RNA was extracted from the normal colon epithelial cells, colon cancer cells, and metastatic cells (i.e., liver metastatic cells) using RNeasy Mini Kit (QIAGEN Inc.), and analyzed with an Experion RNA StdSens chip (Bio-Rad Inc.). For hybridization, biotin-labeled cRNA was prepared using Illumina TotalPrep RNA Amplification kit (Ambion Inc.). Briefly, cDNA of total RNA was synthesized using T7 Oligo(dT) primers, and biotin-labeled cRNA was prepared by in vitro transcription using biotin-UTPs. The cRNA was quantified in NanoDrop. Each cRNA prepared in normal colon epithelial and colon cancer cells was hybridized with Human-6 V2 (Illumina Inc.) chip. After hybridization, DNA chip was washed with Illumina Gene Expression System Wash Buffer (Illumina Inc.) to remove non-specific hybridization, and then labeled with streptavidin-Cy3 fluorescent reagent (Amersham Inc.). The fluorescent-labeled DNA chip was scanned using a confocal laser scanner (Illumina Inc.) to obtain fluorescent data from individual spots, which were stored as TIFF image files. TIFF image files were quantitated with BeadStudio version 3 (Illumina Inc.) for obtaining fluorescent value of individual spots. Quantitative results were normalized using ‘quantile’ function of Avadis Prophetic version 3.3 program (Strand Genomics Ltd.). By analyzing expression profile of genes using a hierarchical clustering analysis, gene expression pattern between normal colon epithelial and colon cancer cells was comparatively determined. Based on results of DNA chip, the present inventors primarily selected genes which are specifically overexpressed in colon cancer and metastasis (FIG. 1). FIG. 2 represented expression pattern of genes overexpressed in colon cancer tissues. Genes listed in FIG. 2 is expressed in colon cancer tissue higher than in normal tissue, and enhanced or similarly expressed in metastatic tissue higher than in colon cancer tissue.

For genes selected primarily, which are overexpressed specifically in colon cancer or metastasis through DNA chip analysis, data mining was performed.

As results, it was demonstrated that ATAD2 gene was specifically overexpressed in colon cancer, enabling to be utilized as a molecular biomarker for colon cancer.

### Example 2

### RT-PCR Analysis of Colon Cancer/Metastasis-Specific Genes

Total forty tissues consisting of twenty samples per each tissues which are extracted from colon cancer tissue, and non-colon cancer tissue adjacent to colon cancer tissue of a colon cancer subject, were frozen in liquid nitrogen. Total RNA was isolated using guanidinium method and stored/used at isopropanol. RNA was quantitated using a spectrophotometer, and checked through SDS-PAGE gel electrophoresis. RT-PCR (reverse transcription-polymerase chain reaction) was carried out to determine whether genes selected from data mining were specifically overexpressed in colon cancer/metastasis. cDNA was synthesized by reverse transcription using RNA extracted from cells of tissues described above. cDNA preparation was performed by AccuAcript High Fielity 1st Stand cDNA synthesis kit (STRATAGENE), followed by PCR using the cDNA products and primers of Table 1. Primers used in PCR amplification were designed using Primer3 Program (http://frodo.wi.mit.edu/) in reference with gene nucleotide sequences from CoreNucleotide of NCBI.

TABLE 1

| | | Product | |
|---|---|---|---|
| | Primer | size (bp) | Sequence |
| 1 | PSAT1-(L) | 356 bp | GTC CAA GCC AGT GGA TGT TT |
| | | | (SEQ ID NO: 1) |
| | PSAT1-(R) | | TAT ACA GAG AGG CCC GGA TG |
| | | | (SEQ ID NO: 2) |
| 2 | ATAD2-(L) | 302 bp | TAT GGA TGG ATT GGA CAG CA |
| | | | (SEQ ID NO: 3) |
| | ATAD2-(R) | | AGA TCT GTG GGT AGC GTC GT |
| | | | (SEQ ID NO: 4) |
| 3 | ASB9-(L) | 303 bp | CAT CAT GGA TGG CAA ACA AG |
| | | | (SEQ ID NO: 5) |
| | ASB9-(R) | | GTC GTC ACA CCA TTC ACC TG |
| | | | (SEQ ID NO: 6) |
| 4 | SLC7A11-(L) | 304 bp | GGC AGT GAC CTT TTC TGA GC |
| | | | (SEQ ID NO: 7) |
| | SLC7A11-(R) | | AAC TGC CAG CCC AAT AAA AA |
| | | | (SEQ ID NO: 8) |
| 5 | CKAP2L-(L) | 327 bp | AAA CGG CCT CCT ATG GAA CT |
| | | | (SEQ ID NO: 9) |
| | CKAP2L-(R) | | TAT TGG TGT TGC CCC ATT TT |
| | | | (SEQ ID NO: 10) |

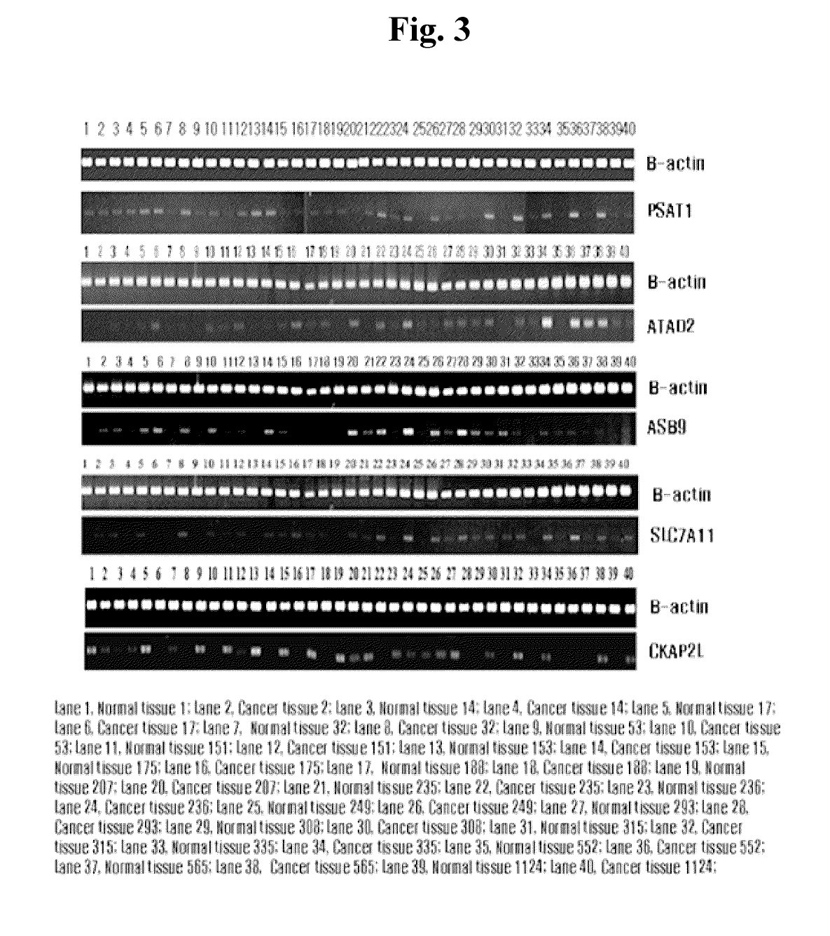

RT-PCR results were shown in FIG. 3. As demonstrated in FIG. 3, it could be appreciated that the biomarkers of the present invention are specifically overexpressed in colon cancer/metastasis tissues. Interestingly, ATAD2 is very specifically overexpressed only in metastatic cells, leading to have most excellent application as a colon cancer metastatic biomarker.

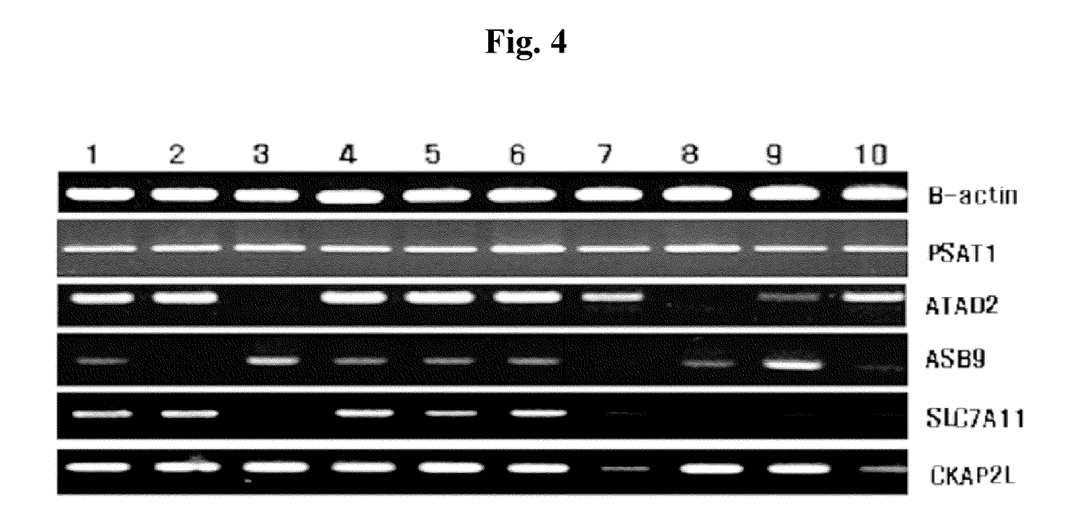

In addition, the expression level of an ATAD2 gene was examined in various colon cancer cell lines (DLD-1, HT29, HCT116, colo205, SW480, SW620, SNU-C1, SNU-C2A, KM12C and KM12SM). As shown in FIG. 4, it could be appreciated that the ATAD2 gene may be very useful as a cancer diagnosis marker due to its high-expression in various colon cancer cell lines.

### Example 3

### Comparison of the Expression Level of Protein in Serum Using Western Blotting

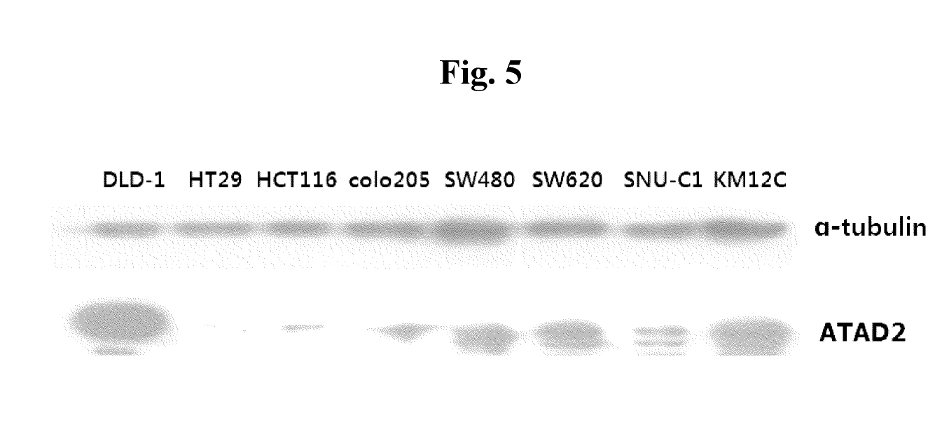

The expression level of an ATAD2 protein was examined in various colon cancer cell lines (DLD-1, HT29, HCT116, colo205, SW480, SW620, SNU-C1 and KM12C). To separate proteins, sample buffer (125 mM Tris pH 6.8, 4% SDS, 10% glycerol, 0.006% bromophenol blue, 1.8% BME) was added to colon cancer cells and boiled for 5 min, followed by electrophoresized on 12% SDS-PAGE. The separated proteins on the SDS-PAGE gel were transferred into a nitrocellulose membrane. The membrane was incubated at TBST solution (10 mM Tris, 100 mM NaCl, 0.05% Tween 20) supplemented with 3% fetal serum albumin for 30 min, and then incubated with an ATAD2 antibody (ABNOVA, 1:2000 dilution) at 4° C. for 2 hrs with stirring. The remaining antibodies were washed with TBST, and the membrane was incubated with a HRP-conjugated secondary antibody (ABCAM, Rabbit polyclonal to Mouse IgG) at 4° C. for 1 hrs with stirring. The images were obtained using solution A (containing luminol and enhancer) and solution B (containing hydrogen peroxide) in a MILLIPORE Corp. ECL kit. As shown in FIG. 5, it may be demonstrated that the ATAD2 protein is highly expressed in most colon cancer cell lines.

### Example 4

### Comparison of the Expression Level of Protein in Tissues by Immunohistochemical Staining

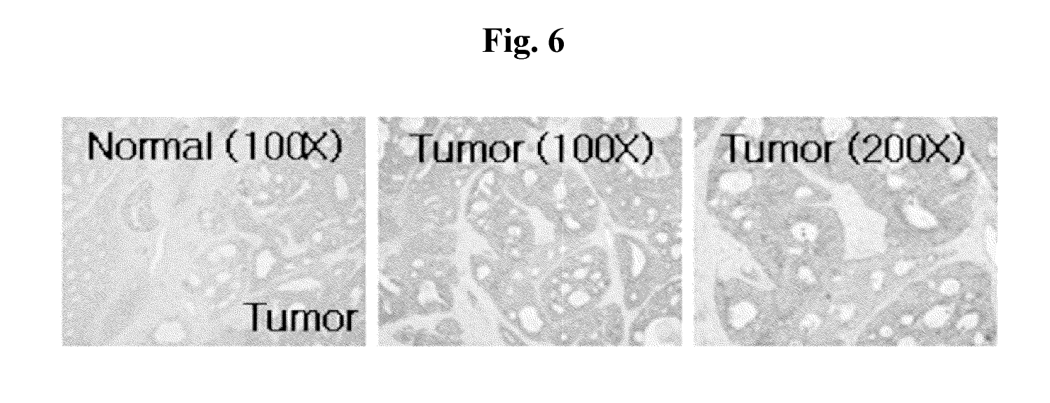

Immunostaining was carried out in a tissue slide to determine the expression pattern and position of ATAD2 protein in normal colon epithelial and colon cancer tissues. Each normal colon epithelial tissues and colon cancer tissues was extracted from colon cancer patients via surgical treatment, and then embedded in a paraffin block. The blocks were sliced at a thickness of 5 μm using a microtome and then attached on a glass slide, preparing tissue slides. The expression pattern and position of ATAD2 protein were observed under a microscope by staining the tissues with ATAD2 antibody (ABNOVA, 1:2000) as described previously. As shown in FIG. 6, it could be appreciated that ATAD2 protein is specifically expressed in colon cancer tissues.

Having described a preferred embodiment of the present invention, it is to be understood that variants and modifications thereof falling within the spirit of the invention may become apparent to those skilled in this art, and the scope of this invention is to be determined by appended claims and their equivalents. All references and documents cited herein are incorporated herein by reference.

What is claimed is:

1. A method for detecting a colon cancer in a human, comprising the steps of:
(a) providing a biological sample from the human; and
(b) detecting the level of a ATAD2 nucleic acid or a ATAD2 protein in the biological sample, relative to the level of the ATAD2 nucleic acid or the ATAD2 protein in a control sample from a normal human, wherein an increased level of the ATAD2 nucleic acid or the ATAD2 protein in the biological sample compared to the control sample indicates that the human has the colon cancer.

2. The method according to claim 1, wherein the detection of the step (b) is carried out by analyzing the level of an mRNA of ATAD2.

3. The method according to claim 2, wherein the analysis of the level of an mRNA of ATAD2 is carried out by a microarray.

4. The method according to claim 2, wherein the analysis of the level of an mRNA of ATAD2 is carried out by RT-PCR (reverse transcription-polymerase chain reaction).

5. The method according to claim 1, wherein the detection of the step (b) is carried out by analyzing the level of the ATAD2 protein.

6. The method according to claim 5, wherein the analysis of the level of the ATAD2 protein is carried out by an immunoassay.

7. The method according to claim 1, wherein the biological sample is a tissue, a cell, blood, serum or plasma.
